(19) United States
(12) Patent Application Publication
(10) Pub. No.: US 2009/0325272 A1
(43) Pub. Date: Dec. 31, 2009

(54) Nucleic Acid Sample Preparation by Exclusion of DNA

(75) Inventors: Abizar A. Lakdawalla, Cupertino, CA (US); David Maurice Cox, Foster City, CA (US)

Correspondence Address: LIFE TECHNOLOGIES CORPORATION;C/O INTELLEVATE, P.O. BOX 52050, MINNEAPOLIS, MN, 55402, US

(73) Assignee: Life Technologies Corporation, Carlsbad, CA (US)

(21) Appl. No.: 12/556,535

(22) Filed: Sep. 9, 2009

Related U.S. Application Data
(63) Continuation of application No. 11/818,979, filed on Jun. 26, 2007.

(30) Foreign Application Priority Data
Jun. 25, 2008 (US) PCT/US08/07899

(51) Int. Cl. C12N 1/08; C12M 1/00

(52) U.S. Cl. 435/270; 435/289.1

(57) ABSTRACT

Devices and methods are provided for separation of nucleic acids from waste materials in a biological sample, and then reacting the separated nucleic acids. In some embodiments, the methods comprise mixing a cell lysate having nucleic acids and waste materials with a waste-binding matrix to capture the cellular waste materials from the lysate. The waste-binding matrix can comprise hydrophilic and/or hydrophobic size-exclusion, ion-exchange particles. In some embodiments, the device comprises a substrate comprising a fluid processing pathway in which nucleic acid sample preparation occurs prior to a downstream genotyping reaction.

### CROSS-REFERENCE TO RELATED APPLICATIONS

The present application is a Continuation of U.S. patent application Ser. No. 11/818,979, filed on Jun. 26, 2007, which is incorporated herein by reference.

### FIELD

The present teachings relate to a method of isolating nucleic acids from a biological sample containing nucleic acids and other materials.

### BACKGROUND

In order to detect a nucleic acid in a biological sample, or to amplify a nucleic acid before detection is required, it is often necessary to separate nucleic acids from other constituents in the sample. Biological samples usually contain large amounts of constituents other than nucleic acids, such as proteins, lipids, and saccharides, which are likely to adversely affect the amplification or detection. Conventional nucleic acid isolation and purification methods require multiple steps and utilize affinity binding of nucleic acids to a matrix, typically, a silica-based matrix. Nucleic acids are bound to the matrix and contaminants are washed off. The nucleic acids are then eluted from the matrix by low salt buffers and heat. As conventional nucleic acid preparation methods require the presence of wash and elution buffer reservoirs and a waste collection chamber, in addition to an affinity capture chamber, it would be difficult to integrate sample preparation and genotyping reactions in a single microfluidic system. A simplified method for isolating and purifying nucleic acids would also be desirable.

### SUMMARY

According to various embodiments, a device is provided having a substrate, a fluid processing pathway is disposed in or on the substrate, an inlet disposed at a first end of the fluid processing pathway, an outlet disposed at a second end of the fluid processing pathway, a reaction chamber disposed along the pathway between the first and second end, and a nucleic acid purification chamber disposed along the pathway between the first end and the reaction chamber. In some embodiments, the reaction chamber can have a pair of forward and reverse polymerase chain reaction primers for together replicating a target nucleic acid sequence. In some embodiments, a polymerase chain reaction product purification chamber can also be disposed along the fluid processing pathway between the reaction chamber and the second end. According to various embodiments, the nucleic acid purification chamber and the polymerase chain reaction product purification chamber can include purification material. The purification material disposed in the nucleic acid purification chamber can comprise a hydrophilic purification material and/or a hydrophobic purification material. According to some embodiments, a second nucleic acid purification chamber can be disposed along the fluid processing pathway between the nucleic acid purification chamber and the reaction chamber. According to some embodiments, first and second nucleic acid purification chambers are provided wherein the first nucleic acid purification chamber comprises a hydrophilic purification material and the second nucleic acid purification chamber comprises a hydrophobic purification material. According to some embodiments, the first nucleic acid purification chamber can comprise a hydrophobic purification material and the second nucleic acid purification chamber can comprise a hydrophilic purification material.

According to some embodiments, the fluid processing pathway can comprise a lysing agent disposed in the fluid processing pathway between the first end and the nucleic acid purification chamber. A sample comprising whole blood cells, for example, can be lysed and purified along the fluid processing pathway. According to some embodiments, the reaction chamber can comprise a magnesium catalyst and/or a polymerase enzyme pre-loaded therein, for example, loaded therein prior to a sample being loaded therein.

According to various embodiments, a method for isolating nucleic acids from cellular waste materials is provided whereby a biological sample is directed into an inlet of a fluid processing pathway and cells in the biological sample are lysed in the fluid processing pathway to form a lysate comprising nucleic acids and other material herein referred to as waste or cellular waste materials. The lysate is then directed into a nucleic acid purification chamber comprising a nucleic acid purification material disposed therein. Nucleic acids that are not captured by the purification material can be directed downstream to a reaction chamber disposed along the fluid processing pathway.

In some embodiments, a protease can be combined with the lysate. In some embodiments, the lysate can be directed by spinning the substrate. The cellular waste material can be captured from the lysate using the nucleic acid purification material, to form purified nucleic acid. The purified nucleic acids can be directed away from the nucleic acid purification chamber.

In some embodiments, the purified nucleic acids can be directed by spinning the substrate. In some embodiments, the purified nucleic acids can be directed by opening a valve along the fluid processing pathway adjacent the nucleic acid purification chamber.

According to some embodiments, the purified nucleic acids can be directed from the nucleic acid purification chamber into a reaction chamber pre-loaded with reaction reagents, for example, with polymerase chain reaction reagents. The reaction reagents can comprise, for example, buffer components, nucleotides, and a pair of primers comprising a forward primer and a reverse primer for together replicating a target sequence of nucleic acids. In some embodiments, the reaction reagents can comprise a polymerase enzyme, a polymerase catalyst, or both.

The nucleic acid purification material can comprise a hydrophilic size-exclusion ion-exchange material. According to some embodiments, the methods can comprise contacting the nucleic acids with hydrophobic size-exclusion particles, for example, before, after, or at the same time that the nucleic acids are contacted with hydrophilic size-exclusion particles.

According to some embodiments, the method can comprise pre-loading in the reaction chamber one or more sequencing primers for replicating a target nucleic acid, that is, for replicating an oligonucleotide comprising a particular sequence of nucleic acids.

These and other embodiments can be more fully understood with reference to the accompanying drawing figures and the descriptions thereof. Modifications that would be recognized by those skilled in the art are considered a part of the present teachings. The drawings are not limiting of the present teachings.

### BRIEF DESCRIPTION OF THE DRAWINGS

FIG. 1 is an illustration of a process for purifying nucleic acids in a biological sample, according to an embodiment of the present teachings.

FIG. 2 is a top view of a fluid processing pathway for processing a nucleic acid-containing sample in a device according to an embodiment of the present teachings;

FIG. 3 is an illustration of an initial step of a method according to an embodiment of the present teachings using the fluid processing pathway of FIG. 2, and showing the pathway in a beginning orientation and containing a loaded sample;

FIG. 4 is a top view of the fluid processing pathway of FIG. 2 and the region of the pathway where sample loading and sealing occurs;

FIG. 5 is a top view of the fluid processing pathway of FIG. 2 and the region of the pathway where polymerase chain reaction occurs;

FIG. 6 is a top view of the fluid processing pathway of FIG. 2 and the region of the pathway where PCR purification occurs;

FIG. 7 is a top view of the fluid processing pathway of FIG. 2 and the region of the pathway where purification and forward and reverse sequencing reactions occur;

FIG. 8 is a top view of the fluid processing pathway of FIG. 2 and the region where fluid communications are formed to open the sequencing reaction chambers and enable purified PCR product to be directed into two respective sequencing chambers;

FIG. 9 is a top view of the fluid processing pathway of FIG. 2 and the region of the pathway where outlets from the sequencing reaction chambers are formed and the sequencing reaction (SR) products are purified in respective sequencing reaction product purification chambers;

FIG. 10 is a top view of the fluid processing pathway of FIG. 2 and the region of the pathway where purified sequencing reaction product from the forward sequencing reaction and from the reverse sequencing reaction are forced into respective product collection wells; and

FIG. 11 depicts the results of gel electrophoresis after PCR amplification of mtDNA from Raji cell lysates treated with different combinations of purification beads.

Other various embodiments of the present teachings will be apparent to those skilled in the art from consideration of the specification and practice of the teachings described herein, and the detailed description that follows. It is intended that the specification and examples be considered as exemplary only.

### DETAILED DESCRIPTION OF CERTAIN EMBODIMENTS

According to various embodiments, the present teachings provide a device and method for separation of nucleic acids from other material, for example, from cellular waste materials such as polypeptides, lipids, salts, short nucleic acid sequences, and carbohydrates. The nucleic acids and other material can be mixed together, for example, in a biological sample.

According to various embodiments, a method is provided whereby cells from a biological sample can be lysed to form a lysate comprising nucleic acids and other material that will be referred to herein as cellular waste materials. The cellular waste material can comprise polypeptides, lipids, carbohydrates, and other material, as described herein.

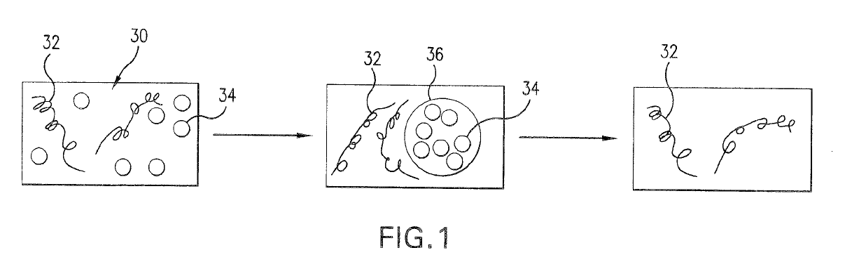

As illustrated in the flowchart of FIG. 1, a cell lysate 30 comprising nucleic acids 32 and cellular waste materials 34 can be mixed with a waste-binding matrix 36, for example, hydrophilic or hydrophobic purification beads, adapted to capture cellular waste materials 34 from lysate 30. Waste-binding matrix 36 can comprise hydrophilic size-exclusion ion-exchange particles and hydrophobic size-exclusion, ion-exchange particles. The size-exclusion, ion-exchange particles can comprise, for example, an ion-exchange core micro-encapsulated by a porous shell, for example, a porous polymeric shell. The shell can be adapted to capture small molecular weight materials and exclude high molecular weight materials. The core can be adapted for ion-exchanging reactions. The nucleic acids that are not captured but instead are left in solution can then be directed downstream for subsequent processing and/or analysis. No washing or elution steps are required, as the nucleic acids do not become bound by or otherwise incorporated in or with waste binding matrix 36.

Exemplary size-exclusion, ion-exchange particles that can be used according to various embodiments are described, for example, in U.S. Patent Application Publication No. US2006/0160122, published Jul. 20, 2006, U.S. Patent Application Publication No. US2006/0051583, published Mar. 9, 2006, U.S. Patent Application Publication No. US2005/0196856, published Sep. 8, 2005, U.S. Patent Application Publication No. US2005/0181378, published Aug. 18, 2005, and U.S. Patent Application Publication No. US2004/0018559, published Jan. 29, 2004, all of which are herein incorporated in their entireties by reference. An exemplary molecular weight cut-off of the molecules that are captured as opposed to those that are not captured can be any suitable cut-off, for example, 500 atomic units or 1500 atomic units, based on weight average molecular weight.

In some embodiments, the biological sample that can be purified according to the present teachings can comprise animal-derived biological material such as blood, urine, saliva, or the like body fluid, or a material derived from organisms other than animals, for example, derived from plants or microorganisms. The biological sample can comprise tissue homogeneates, cells, cell lysates derived from organisms, cell cultures, or partially purified nucleic acids.

According to various embodiments, the nucleic acids that can be purified can comprise deoxyribonucleic acids (DNA) or ribonucleic acids (RNA), for example, double-stranded DNA, single-stranded DNA, plasmid DNA, genomic DNA, cDNA, RNA, RNA derived from exogenous parasites such as viruses, bacteria and fungi, endogenous RNA derived from organisms, tRNA, mRNA, rRNA, siRNA, and combinations thereof.

Although purification with the purification materials described herein can occur in a reaction chamber disposed along a fluid processing pathway in a substrate, it is to be understood that the purification can also be conducted by contacting a cell lysate with the purification material in a reaction chamber that is not disposed along a fluid processing pathway in or on a substrate. For example, the purification can occur in a test tube, beaker, or other separate container prior to loading a resultant purified sample into a PCR reaction chamber.

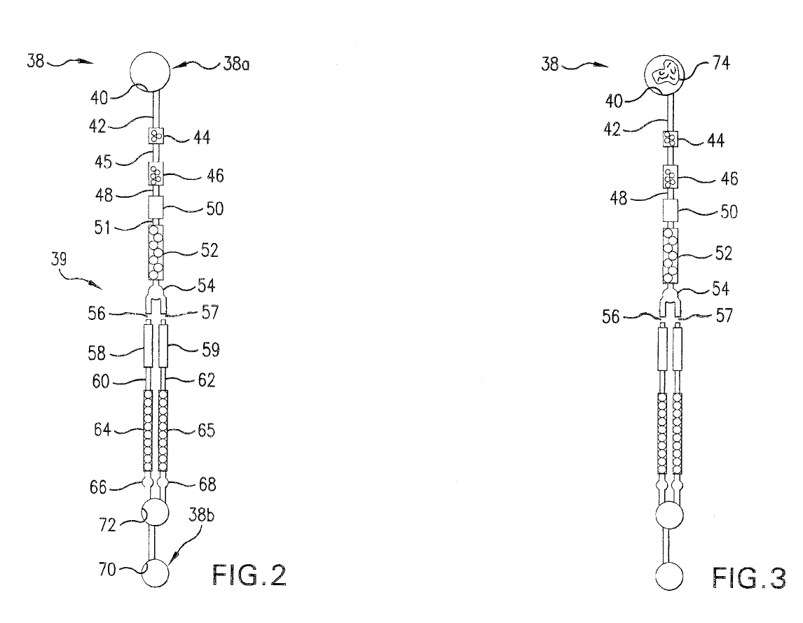

FIG. 2 is a top view of a fluid processing pathway 38 of a fluid processing assembly 39 for processing a biological sample, according to various embodiments. Exemplary fluid processing assemblies sharing some similar features as fluid processing assembly 39 are described in detail, for example, in U.S. patent applications Ser. Nos. 10/336,706 and 10/336,330, filed Jan. 3, 2003, and in U.S. Pat. No. 7,201,881 B2, issued Apr. 10, 2007, U.S. Pat. No. 7,198,759 B2, issued Apr. 3, 2007, U.S. Pat. No. 6,935,617 B2, issued Aug. 30, 2005, and U.S. Pat. No. 7,135,147 B2, issued Nov. 14, 2006, all of which are herein incorporated in their entireties by reference.

A sample can be processed using assembly 39 and the various method steps illustrated sequentially in FIGS. 3-10. As illustrated in FIGS. 3-10 and discussed below, nucleic acid sample preparation and genotyping reactions can be integrated in a single fluid processing assembly.

As illustrated in FIGS. 2-10, exemplary fluid processing pathway 38 comprises an input chamber 40, a fluid communication 42 between input chamber 40 and a first nucleic acid purification chamber 44, a fluid communication 45 between chamber 44 and a second nucleic acid purification chamber 46, a fluid communication 48 between chamber 46 and a reaction chamber 50, a reaction product purification chamber 52, and a fluid communication 51 between chambers 50 and 52. Also shown in FIGS. 2-10, fluid processing pathway 38 comprises a flow splitter 54 in fluid communication with reaction product purification chamber 52, through a fluid communication 53. A forward sequencing reaction chamber 58 and a reverse sequencing reaction chamber 59 can be in fluid communication with flow splitter 54 through respective fluid communications that are generated when a first valve 56 and a second valve 57, respectively, are opened.

Downstream of sequencing reaction chambers 58 and 59 are sequencing reaction product purification chambers 64 and 65 which are respectively in fluid communication with chambers 58 and 59 through fluid communications 60 and 62, respectively. Downstream of purification chambers 64 and 65 are output chambers 70 and 72, respectively, which are respectively in fluid communication with chambers 64 and 65 through fluid communications 66 and 68, respectively. Herein the term “in fluid communication” refers to two features that are in communication with one another by a channel, opening, and/or valve, even if the communication comprises a valve in a closed state but provided that the valve can be opened whereby a fluid can be moved from one of the features to the other.

FIG. 3 is an illustration of an initial step of a method using assembly 39 of FIG. 2. Pathway 38 is depicted in an initial state and containing a sample 74 loaded in input chamber 40.

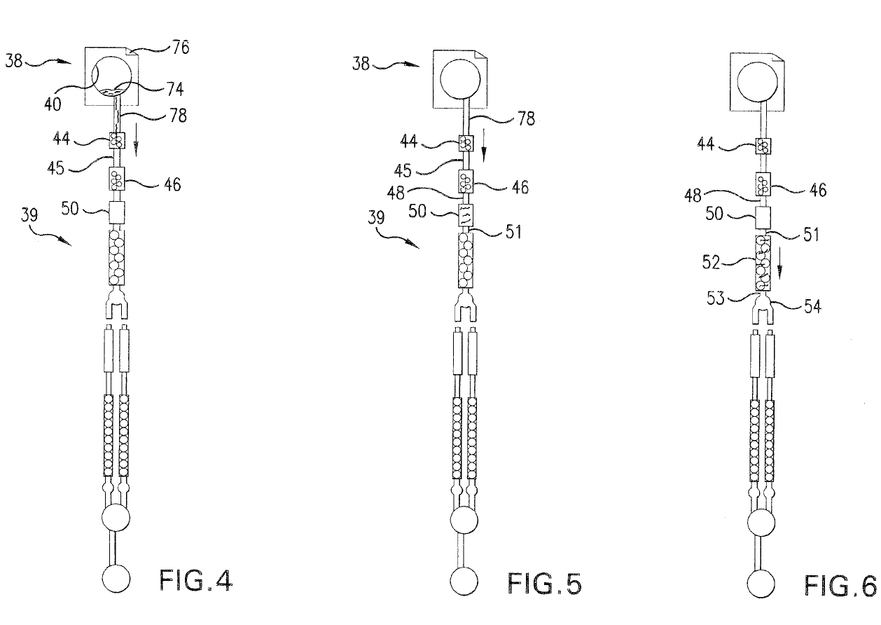

FIG. 4 is a top view of pathway 38 after sample loading and sealing has occurred. As shown in FIG. 4, an adhesive seal 76 has been placed over the open upper end of input chamber 40 after sample 74 has been loaded into input chamber 40. FIG. 4 also shows, by way of the unlabeled directional arrow, the direction of movement of the sample from input chamber 40 into first nucleic acid purification chamber 44. Movement of the sample can be facilitated, for example, by any appropriate force, for example, by gravity, centripetal force, capillary action, pressure differential, or the like. In the exemplary embodiment depicted in FIGS. 2-10, movement of fluid through the device can be facilitated by centripetal force, for example, as provided by spinning a substrate in which or on which fluid processing pathway 38 is formed. Exemplary systems and methods for moving fluid samples through a processing pathway are shown, for example, in U.S. Pat. No. 7,198,759 B2, issued Apr. 3, 2007, which is incorporated herein in its entirety by reference.

The volume of the first nucleic acid purification chamber can be large enough to contain an entire sample that is input into input chamber 40 while also containing purification material. As described herein, the purification material can comprise hydrophilic and/or hydrophobic purification particles or beads. In some embodiments, the sample can be directed into first nucleic acid chamber 44 but be prevented from passing into second nucleic acid purification chamber 46 by way of incorporating a valve in a closed position along fluid communication 45. Any appropriate valve in a closed position can be provided, for example, a heat-meltable valve, a dissolvable valve, or a deformable valve. Exemplary deformable valves that can be used include those described, for example, as Zbig valves in U.S. Pat. No. 7,198,759 B2.

After contacting the sample with purification material in nucleic acid purification chamber 44 for an appropriate period of time, the valve along fluid communication 45 can be opened and a sample movement force can be generated to direct the sample from nucleic acid purification chamber 44 into second nucleic acid purification chamber 46. Further movement of the purified sample through fluid processing pathway 38 can be prevented by providing an appropriate valve along fluid communication 48, for example, the same type of valve as used along fluid communication 45.

The residence time or contact time in each of nucleic acid purification chambers 44 and 46 can be the same or different from one another. In some embodiments, a hydrophilic purification material can be pre-loaded in nucleic acid purification chamber 44 and a hydrophobic purification material can be pre-loaded in nucleic acid purification chamber 46. The contact time can be, for example, at least fifteen seconds, at least thirty seconds, at least forty-five seconds, at least one minute, or at least two minutes, according to various embodiments. In some examples, the contact time can be from forty-five seconds to about sixty seconds or from about thirty seconds to about ninety seconds, independently, in each of first and second nucleic acid purification chambers 44, 46.

FIG. 5 is a top view of pathway 38 and the region of the pathway 38 where a first reaction with the purified sample can occur. The first reaction be comprise, for example, a polymerase chain reaction in reaction chamber 50. In the embodiment shown in FIG. 5, the sample has been purified in both first nucleic acid purification chamber 44 and second nucleic acid purification chamber 46. A valve disposed along fluid communication 48 has been opened and the sample has been moved from second nucleic acid purification chamber 46 through fluid communication 48 and into reaction chamber 50. At this point in the method depicted, the valve disposed along fluid communication 48 can be reclosed, and a closed valve can be provided along fluid communication 51 downstream of reaction chamber 50. An exemplary reclosable valve that can be used along fluid communication 48 can comprise a reclosable valve as described, for example, in U.S. Pat. No. 6,935,617 B2, which is incorporated herein in its entirety be reference.

Once the valves immediately upstream and immediately downstream from reaction chamber 50 are closed, thermal processing or thermocycling of the contents of reaction chamber 50 can be conducted, for example, as is useful to promote polymerase chain reaction between the purified sample in reaction chamber 50 and PCR reagents that had been pre-loaded in reaction chamber 50. For such purposes, reaction chamber 50 can be pre-loaded, for example, with magnesium catalyst, forward and reverse PCR primers, polymerase enzyme, nucleotide triphosphates, and like reagents useful for conducting polymerase chain reaction. In some embodiments, the pre-loaded reagents can comprise reagents that might otherwise be captured upstream of reaction chamber 50 by purification material in either or both of nucleic acid purification chambers 44 and 46. Systems and methods for carrying out thermocycling of the contents of reaction chamber 50 are described, for example, in U.S. Pat. No. 7,198,759 B2, and in U.S. Patent Application Publication Nos US 2006/0046304 A1 (Mar. 2, 2006), and US2006/0239666 A1 (Oct. 26, 2006).

After thermocycling, the valve disposed along fluid communication 51 can be opened, for example, by a deforming action, and the PCR product generated by thermocycling the contents of reaction chamber 50 can be moved in the direction shown by the directional arrow into reaction product purification chamber 52. The PCR product can be made to reside in reaction product purification chamber 52, and to thus contact purification material disposed therein, for a period of time. The period of time can be at least fifteen seconds, at least thirty seconds, at least forty-five seconds, at least sixty seconds, at least two minutes or for a period of time within a range of from about thirty seconds to about ninety seconds, or from about forty-five seconds to about sixty seconds. The reaction product purification material can comprise the size-exclusion, ion-exchange particles described herein, for example, those described in U.S. Patent Application Publication No. US2006/0160122, which is incorporated herein in its entirety by reference. The PCR product can be prevented from passing downstream of reaction product purification chamber 52 by providing a closed valve along fluid communication 53 between reaction product purification chamber 52 and flow splitter 54.

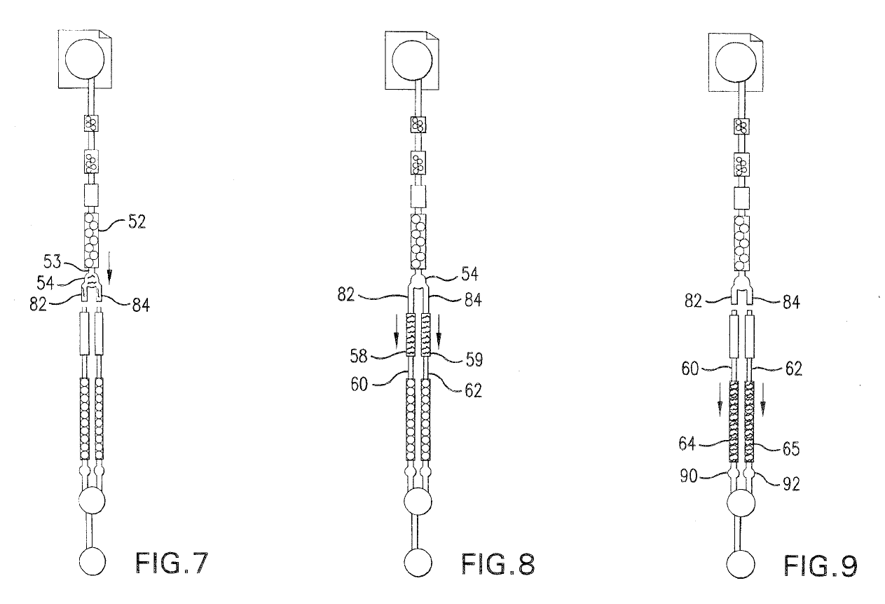

After an appropriate residence time in reaction product purification chamber 52, the valve disposed along fluid communication 53 can be opened, as shown in FIG. 7, and the purified PCR product can be moved into flow splitter 54, again, for example, by centripetal force. After the purified PCR product is moved into flow splitter 54, valves along fluid communications 82 and 84 can be respectively opened and the purified PCR product can be made to flow into sequencing reaction chambers 58 and 59, respectively, as shown in FIG. 8. As shown in FIG. 8, the purified PCR product can be moved in the direction shown by the directional arrows by using centripetal force as described herein.

Movement of the purified PCR product downstream of sequencing reaction chambers 58 and 59 can be prevented by providing closed valves along downstream fluid communications 60 and 62, respectively. After the purified PCR product is moved into sequencing reaction chambers 58 and 59, the valve along fluid communications 82 and 84 can be closed and thermal processing or thermocycling can be conducted on the purified PCR products in sequencing reaction chambers 58 and 59. Sequencing reaction chambers 58 and 59 can be pre-loaded with sequencing reaction reagents, for example, that can be dried down after pre-loading and then solubilized by the solution that contains the purified PCR product. In some embodiments, flow splitter 54 can be designed such that half of the purified PCR product from inside flow splitter 54 can be moved along fluid communication 82 to sequencing reaction chamber 58, and the other half of the purified PCR product can be moved through fluid communication 84 into sequencing reaction chamber 59.

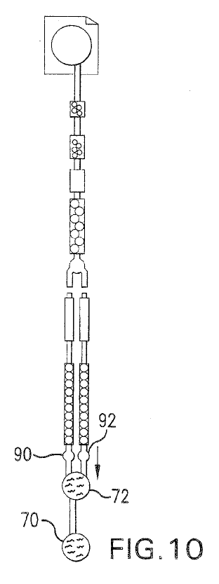

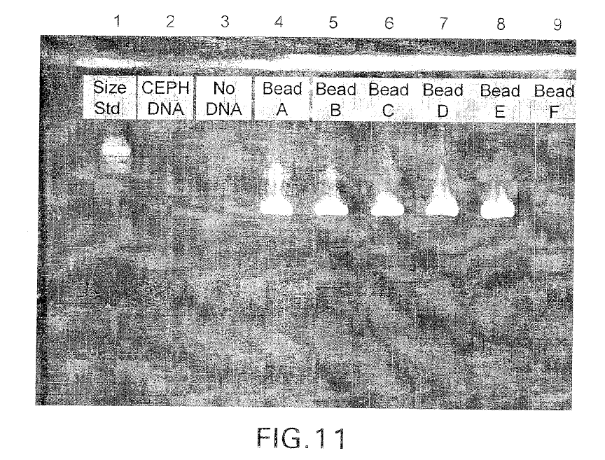

After sequencing reactions occur in sequencing reaction chambers 58 and 59, the valves along fluid communications 60 and 62 can be opened and the sequencing reaction products can be moved through fluid communications 60 and 62 into sequencing reaction product purification chambers 64 and 65, respectively. The sequencing reaction products can be purified in sequencing reaction product purification chambers 64 and 65 for a period of time. The period of time can be, for example, at least fifteen seconds, at least thirty seconds, at least forty-five seconds, at least sixty seconds, at least two minutes, or within the range of from about thirty seconds to about ninety seconds, or from about forty-five seconds to about sixty seconds. The sequencing reaction products can be prevented from moving downstream of sequencing reaction product purification chambers 64 and 65 by providing closed valves along fluid communications 90 and 92 downstream of sequencing reaction product purification chambers 64 and 65, respectively. After the appropriate contact time with purification material in sequencing reaction product purification chambers 64 and 65, the valves along fluid communications 90 and 92 can be opened and the purified sequencing reaction products can be moved into output chambers 70 and 72, as shown in FIG. 10. The purified forward sequencing reaction products and the purified reverse sequencing reaction products can subsequently be removed from output chambers 70 and 72 for further processing, for example, further processing involving capillary electrophoresis.

Although a method comprising DNA isolation followed by PCR is detailed above, it is to be understood that pre-PCR purification of other components, and other post-PCR processing steps, can be conducted while remaining within the scope of the present teachings. For example, in some embodiments, a DNA sequencing method can be carried out whereby cells are reacted with a lysis buffer containing enzymes to digest proteins and RNA, followed by capture of non-DNA materials using the purification materials described herein, followed by PCR amplification of the purified DNA, followed by purification, followed by one or more sequencing reactions, followed by one or more other purification steps.

In some embodiments, an RNA sequencing method can be carried out whereby cells are reacted with a lysis buffer containing enzymes to digest proteins and DNA. The method can comprise following lysis with the capture of non-RNA materials using the purification materials described herein. Then, the purified RNA can be subjected to reverse transcription and PCR amplification, followed by purification. Then, the product can be subjected to one or more sequencing reactions, followed by one or more other purification steps.

In some embodiments, a qPCR-based genotyping method can be carried out whereby cells are reacted with a lysis buffer containing enzymes to digest proteins and RNA. The method can comprise following lysis with the capture of non-DNA materials using the purification materials described herein. Then, the purified DNA can be subjected to real-time PCR amplification, followed by readout/analysis.

In some embodiments, a qPCR-based gene expression method can be carried out whereby cells are reacted with a lysis buffer containing enzymes to digest proteins and DNA. The method can comprise following lysis with the capture of non-RNA materials using the purification materials described herein. Then, the purified RNA can be subjected to real-time PCR amplification, followed by readout/analysis. In some embodiments, the purified RNA can be subjected to reverse transcription and then real-time PCR amplification, followed by readout/analysis.

Although the examples below show the use of specified volume ratios of certain types of purification materials in mixtures, it is to be understood that any of a variety of ratios can be used. For example, volume ratios of from one part hydrophilic purification material to ten parts hydrophobic material, to ten parts hydrophilic material to one part hydrophobic material, can be used, for example, volume ratios of from one part to five to five parts to one, and volume ratios of about one to one, can be used.

### EXAMPLES

### Example 1

Tissue culture cells were grown and resuspended in a lysis buffer. The lysis buffer included a detergent that broke down the cellular structure (dissolved lipids). The lysis buffer also included a protease that broke down the proteins into smaller peptides and amino acids resulting in the release of DNA and RNA. For reactions involving a lysis buffer that contained RNAse, RNA was also removed from the mix, leaving undegraded DNA.

Unmodified beads were used to demonstrate that cell components, other than highly charged high-molecular weight molecules like genomic DNA and mRNAs, are adsorbed by beads that bind to hydrophilic and hydrophilic small molecular weight cellular components. A mixture of the following three types of beads was used:
SEPHADEX G-50—traps small molecular weight charged components (nucleotides, small sugars, salts, ions, peptides, amino acids). (available from Sigma-Aldrich Co., St. Louis, Mo., Cat. No. G50300, CAS No. 9048-71-9);
SEPHAROSE 4B—traps larger components, including peptides, carbohydrates, oligonucleotides (available from Sigma-Aldrich Co., Cat. No. 4B200, CAS No. 9012-36-6); and
SEPHADEX LH 20—traps lipids, steroids, fatty acids, hormones, and vitamins (available from Sigma-Aldrich Co., Cat. No. LH20100).

### Example 2

Raji cells were grown to confluence in standard culture medium with Fetal Calf serum. Cells were removed from the tissue culture dish having a trypsin treatment. Released cells were collected by centrifugation. The cell pellet was resuspended in 5 ml of Phosphate Buffered Saline and re-centrifuged. The cell pellet was resuspended in lysis buffer (at approximately 5000 cells/ul) containing a strong detergent and the pronase protease. The cells were digested at 37° C. for 15 min in 100 uL volumes resulting in the dissolution of lipid structures and of proteins. A 50 ul mixture of beads were added to 100 ul of cell lysates and incubated at room temperature with intermittent mixing for 10 minutes. Different combinations of the following types of beads were used:
OCTYL SEPHAROSE CL-4B (Sigma-Aldrich)—to capture small molecular weight lipids, steroids, fatty acids, hormones, and hydrophobic substances in the lysate;
SEPHAROSE 4B—to capture peptides; and
SEPHADEX G50—to remove salts and small molecular weight biomolecules.

The bead/lysate mixture was centrifuged and an aliquot of the supernatant was used for PCR amplification. PCR amplification was done with mitoSEQr primers (against human mtDNA) using 0.6 uM of primers in 2× Fast PCR master mix with 2 ul of gDNA. Cycling conditions comprised heating to 95° C. for one minute followed by 40 cycles of fast PCR. Each cycle of fast PCR comprised heating the sample at 95° C. for five seconds, then heating the sample at 60° C. for 20 seconds, followed by heating the sample at 72° C. for 25 seconds.

PCR products were run on a 1% agarose gel containing ethodium bromide. The gel was imaged. FIG. 11 depicts the results of the gel electrophoresis. As shown in FIG. 11, successful PCR amplification was seen with most combinations of the beads. The combinations differed in the proportion of each type of beads. Bead combination A comprised one volume of SEPHADEX G50, one volume of SEPHAROSE 4B, and one volume of OCTYL SEPHAROSE CL-4B, mited together. Bead combination B comprised one volume of SEPHADEX G50, six volumes of SEPHAROSE 4B, and three volumes of OCTYL SEPHAROSE CL-4B. Bead combination C comprised six volumes of SEPHADEX G50, one volume of SEPHAROSE 4B, and three volumes of OCTYL SEPHAROSE CL-4B. Bead combination D comprised seven volumes of SEPHADEX G50, no SEPHAROSE 4B, and three volumes of SEPHAROSE CL-4B. Bead combination E comprised no SEPHADEX G50, seven volumes of SEPHAROSE 4B, and three volumes of OCTYL SEPHAROSE CL-4B. Bead combination F comprised one volume of SEPHADEX G50, one volume of SEPHAROSE 4B, and no SEPHAROSE CL-4B

Those skilled in the art can appreciate from the foregoing description that the present teachings can be implemented in a variety of forms. Therefore, while these teachings have been described in connection with particular embodiments and examples thereof, the present teachings should not be so limited. Various changes and modifications can be made without departing from the scope of the present teachings.

What is claimed:

1. A device comprising:
a substrate; and
a fluid processing pathway disposed in or on the substrate having a length and comprising
a first end and a second end,
an inlet disposed at the first end,
an outlet disposed at the second end,
a reaction chamber disposed along the pathway between the first end and the second end and comprising a pair of forward and reverse polymerase chain reaction primers for together replicating a target nucleic acid sequence,
a nucleic acid purification chamber disposed along the pathway between the first end and the reaction chamber, and
a polymerase chain reaction product purification chamber disposed along the fluid processing pathway between the reaction chamber and the second end,
wherein the nucleic acid purification chamber comprises a nucleic acid purification material disposed therein and the polymerase chain reaction product purification chamber comprises purification material disposed therein.

2. The device of claim 1 further comprising a lysing agent disposed in the fluid processing pathway between the first end and the nucleic acid purification chamber.

3. The device of claim 1, wherein the reaction chamber further comprises a magnesium catalyst pre-loaded therein.

4. The device of claim 1, wherein the reaction chamber further comprises a polymerase enzyme pre-loaded therein.

5. The device of claim 1, wherein the purification material disposed in the nucleic acid purification chamber comprises hydrophilic purification beads and hydrophobic purification beads.

6. The device of claim 1, wherein the purification material disposed in the nucleic acid purification chamber comprises hydrophilic purification beads and hydrophobic purification beads in a ratio of from about one to five to about five to one, respectively, by volume.

7. The device of claim 1, further comprising a second nucleic acid purification chamber disposed along the fluid processing pathway between the nucleic acid purification chamber and the reaction chamber.

8. The device of claim 7, further comprising a hydrophilic purification material disposed in the nucleic acid purification chamber and a hydrophobic purification material disposed in the second nucleic acid purification chamber.

9. The device of claim 7, further comprising a hydrophobic purification material disposed in the nucleic acid purification chamber and a hydrophilic purification material disposed in the second nucleic acid purification chamber.

10. A method for isolating nucleic acids from cellular waste materials comprising:
directing a biological sample comprising one or more cells into an inlet of a fluid processing pathway;
lysing the one or more cells in the biological sample to form a lysate comprising nucleic acids and cellular waste materials;
contacting the lysate with a nucleic acid purification material that is disposed within a nucleic acid purification chamber;
capturing the cellular waste materials with the nucleic acid purification material, to form purified nucleic acids; and
directing the purified nucleic acids from the nucleic acid purification chamber into a reaction chamber pathway pre-loaded with reaction reagents.

11. The method of claim 10, wherein the nucleic acid purification material comprises a hydrophilic size-exclusion ion-exchange material.

12. The method of claim 10, further comprising contacting the purified nucleic acids with hydrophobic size-exclusion particles.

13. The method of claim 10, further comprising combining a protease with the lysate.

14. The method of claim 10, wherein the pre-loaded reaction reagents comprise polymerase chain reaction reagents.

15. The method of claim 10, wherein the pre-loaded reaction reagents comprise a pair of forward and reverse polymerase chain reaction primers for together replicating a target nucleic acid sequence.

16. The method of claim 10, further comprising pre-loading the reaction chamber with a polymerase enzyme.

17. The method of claim 10, wherein the directing the lysate comprises spinning the substrate.

18. The method of claim 10, wherein the directing the purified nucleic acids comprises spinning the substrate.

19. The method of claim 10, wherein the directing the purified nucleic acids comprises opening a valve along the fluid processing pathway adjacent the nucleic acid purification chamber.

20. The method of claim 10, wherein the reaction chamber is disposed along the fluid processing pathway.
